(19) United States
(12) Patent Application Publication
(10) Pub. No.: US 2014/0031608 A1
(43) Pub. Date: Jan. 30, 2014

(54) Psychotherapeutic Process for Mitigation of Chronic Afflictions

(71) Applicant: Robert Osann, JR., Port Angeles, WA (US)

(72) Inventor: Robert Osann, JR., Port Angeles, WA (US)

(21) Appl. No.: 13/557,155

(22) Filed: Jul. 24, 2012

(51) Int. Cl. A61M 21/02

(52) U.S. Cl. 600/26

(57) ABSTRACT

It is generally understood that psychogenic afflictions exist, and their study forms the basis of psychosomatic medicine. Psychogenic pain and other afflictions serve the purpose of diverting a subject's thoughts away from thoughts they find painful or unbearable. As such, a subject frequently is not aware of what they were thinking just prior to the onset of an affliction—so thoroughly does the body's psychogenic distraction mechanism work. As disclosed herein, a posthypnotic process is imparted to a subject under hypnosis to uncover whatever painful thoughts the subject has immediately preceding the onset a psychogenic affliction. The painful thoughts may be immediately written down by the subject or remembered, either consciously or unconsciously, to be revealed later to a therapist. An alternate form of posthypnotic process includes thoughts and actions that a subject is encouraged to use in mitigating the affliction.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

This invention relates to the field of psychoanalysis and its application to the management and relief of chronic afflictions, and to the use of hypnosis on subjects as part of a psychotherapeutic process.

### BACKGROUND

First, a word of caution. No one should assume that his or her symptoms are psychologically caused until a physician has ruled out the possibility of serious or life-threatening disease. Given that caution, it is generally acknowledged that many chronic afflictions are psychogenic.

Conventional medicine focuses on physical solutions for physical afflictions. One reason for this is the impatience of the general populace, especially in current times where the pace of life is so rapid and complex. A pill to render a quick solution is usually the desired solution. For chronic joint pain, surgery is frequently sought on doctor's advice and especially where the patient is desperate. One study showed that over $25B is spent annually in the US alone for treating back pain. Most back surgery patients are not aware that there may be alternative treatments for their pain, especially where their pain is chronic and intermittent. They also are probably not aware that the cause of their pain may not in fact be the anomaly they were shown on an MRI image.

Mindbody medicine is still in its infancy even though the mindbody connection has been known for decades. When chronic pain sufferers are told that their pain may be “psychosomatic”, they typically back away. One reason is that the term “psychosomatic” has been made synonymous with “it's all in your mind”, or in other words insinuating that the pain is imagined. In reality, a better term for the very real physical pain that results from mindbody interaction is “psychogenic”, in other words real pain caused by the mind. One might ask: “How can the mind generate pain, and why should the mind do this?” Here, the Books of Dr. John Sarno are probably the authority on the subject and provide the best explanation.

Dr. Sarno's books include “Mind Over Back Pain”; “Healing Back Pain: The Mind-Body Connection”; and “The Mindbody Prescription: Healing the Body, Healing the Pain”. Dr. Sarno describes the process of psychogenic pain (and other psychogenic afflictions) and has generically named the phenomenon TMS or Tension Myositis Syndrome. Here, the mind controls an area of the body to cause pain by controlling localized circulation or through other physiological methods over which the mind has influence. While we don't commonly think about the body's ability to control circulation and localized body chemistries, the literature points to the body's ability to adapt and cites various phenomenon such as for example the “fight or flight” response. When this mechanism is activated, certain muscles receive an abnormally large share of circulation while others (such as those used in digestion) receive very little. In other words, the body's ability to control functions such as circulation also provide it a means to control those functions to generate pain or other afflictions, should the mind so choose.

One might then ask: “Why would the body choose to generate pain?” The very simple answer is: “to distract the subject from thinking about something they find unpleasant, painful, or unbearable”. The literature discusses in great depth the reasons for psychosomatic pain generation, including some very deep and complex Freudian concepts and theories. That aside, the present invention views the body's auto-generation of chronic afflictions as an automated distraction mechanism—plain and simple. First, a subject thinks about something unpleasant, painful, or unbearable—that thought process occurring either consciously or unconsciously. Second, the subject's mind creates a physical distraction mechanism so painful, distracting, or unpleasant in itself, that the mind is immediately distracted from the painful thoughts. It's as though the mind has decided at an unconscious level that the lesser of two evils is to replace painful thoughts with real afflictions such as for example pain. While Dr. Sarno describes the culprit as “Unconscious Rage and Unbearable Feelings”, the term “Painful Thoughts” is hereinafter used generically to describe any unpleasant, painful, or unbearable thoughts that result in the onset of an episode of a psychogenic affliction in a subject.

Who Suffers from Psychogenic Afflictions

This discussion has two parts: What personality types are prone to psychogenic afflictions, and how a physician can determine that a subject is most likely to have such afflictions manifested. Dr. Sarno identified a personality type as being more prone to psychogenic afflictions. This personality type included the following characteristics: hardworking; conscientious; responsible; compulsive; and perfectionistic. In addition, the more a subject has exhibited any of a list of known psychogenic afflictions in the past, the more likely they are to exhibit others in the present and future.

Chronic afflictions that have well known and widely recognized mindbody connections include: indigestion and ulcers; skin diseases (hives, rosacea, lichen planus, and seborrhea psoriasis); asthma; and hypertension. Also, it is well recognized that many people may have a “nervous stomach” where they become nauseous and even vomit in stressful situations—certainly a mindbody phenomenon. According to some sources, it has been more recently determined that mindbody afflictions also include: irritable bowel syndrome; joint pain—back, neck, shoulder, knee, hip, etc; allergies; fibromyalgia; osteoarthritis; tinnitus, migraines; swollen prostate; and gout.

For many of these, conventional medicine looks for structural anomalies, especially for joint pain, or to external and physical triggers, like chocolate or fragrances that supposedly trigger migraines, or pollen that triggers allergies. The reality is that a major contributor and sometimes the sole cause of chronic afflictions are painful thoughts that the mind decides must be diverted through some form of distraction. A thorough and effective remedy for these chronic afflictions must deal with this mindbody phenomenon.

Testing for Propensity Toward Psychogenic Afflictions

In addition to enumerating any mindbody afflictions a subject has exhibited in the past, the following method quoted from Dr. Sarno's book: “The Mindbody Prescription: Healing the Body, Healing the Pain” describes how a practitioner can determine that an individual is prone to certain forms of psychogenic affliction:
“On physical examination, almost every [TMS] patient was found to have a tenderness on palpation of (or pressing on) certain muscles regardless of where in the neck or back they felt pain. For example, someone might have pain only in the right lower back but on examination felt pain when I pressed on the top of both shoulders (upper trapezius muscles), the small of the back on both sides (lumbar paraspinal muscles), and the outer part of both buttocks (gluteal muscles). This consistent finding strongly suggested that the syndrome originated in the central nervous system (brain) rather than in a local structural abnormality.

Conscious, Subconscious, and Unconscious Thought

Much literature exists discussing the nature of conscious, subconscious, and unconscious thought, and numerous discussion threads exist online where the subtleties and differences of these are debated. Overall, there appears to be no consensus regarding the differences between subconscious and unconscious thought processes except that they are different from thought that a subject is consciously aware of Hence regarding descriptions herein and appended claims, it is assumed that subconscious thought and unconscious thought are essentially the same and are herein referred to simply as “unconscious” thought.

Magnitude of the Problem

In spite of the general increase in awareness regarding mindbody phenomenon as relates to chronic afflictions, there has been little attention paid to this issue among the medical community at large. As a result, society annually spends billions of dollars on medical procedures and surgeries that are essentially unnecessary—frequently having little or no effect on a subject's affliction or sometimes providing a temporary placebo result, while at the same time putting some subjects at risk, especially in cases where surgery is performed. As one prominent Bay Area neurosurgeon replied when asked by a post-operative patient about a specific manifestation of back pain: “The causes of back pain really are not well understood”.

### SUMMARY

The purpose of psychogenic pain and most psychogenic afflictions is to distract a subject from unpleasant or painful thoughts. As such, when an episode, event, or onset of an affliction occurs, the subject typically forgets the thoughts that were in their mind just a few seconds prior to the onset of the affliction. The mechanism works very well, and in some cases, the intensity or severity of the affliction may be proportional to the intensity of distress embodied in the painful thoughts. Psychogenic pain and affliction according to this invention include for example but are not limited to back pain, neck pain, joint pain, muscle pain, tendon pain, numbness, fibromyalgia, digestive problems, and migraine headaches including optical migraines where no pain is present. Also, some psychogenic afflictions may not be manifested physically and instead may constitute a mental affliction such as for example depression or certain afflictions associated with PTSD. While depression is known in some cases to be related to chemical imbalances in the brain, the psychogenic contribution to an episode of depression is sometimes downplayed. To overcome a psychogenic affliction, it is necessary to access and address the painful thoughts in one or more ways. Therefore, the painful thoughts must be located which is directly counter to what the mind and body accomplish by generating the affliction. Also, the painful thoughts may actually be either conscious or unconscious, or be some mixture of the two. If the painful thoughts are unconscious, only a method like a hypnosis-induced unconscious process can access unconscious thoughts that precede the onset of an episode of an affliction, and enable the related issues to be revealed and subsequently addressed. While some subjects with coaching are able to consciously overcome their mind and body's distraction mechanism and locate conscious painful thoughts, many subjects either cannot or will not. If the painful thoughts are unconscious only, no subject will be able to consciously locate them without the help of mechanisms such as those described herein that operate at least in part in the unconscious mind.

Some subjects, with proper hypnotic induced guidance will be able to think back in time upon an onset of the affliction. Even if their conscious mind has quickly forgotten the painful thoughts, their unconscious mind still remembers, and a post-hypnotic process (hereinafter: “posthypnotic process”) as described herein will enable them to locate those thoughts. Some subjects may not be able to accomplish this consciously even with hypnotic induced guidance—however since their unconscious mind can locate and remember the thoughts, it is possible for an alternate form of posthypnotic process to cause the locating and remembering of the painful thoughts to happen unconsciously. Either way, in a subsequent hypnotic session, a therapist can uncover the painful thoughts and assist the subject in dealing with them. Given the psychological nature of what may be uncovered by the processes described herein, it is preferred that the therapist is also a qualified mental health councilor in addition to a hypnotist.

Posthypnotic Process and Triggers

According to the invention, a posthypnotic process is a process involving a subject that occurs after the conclusion of a hypnosis session with the subject, and wherein the process incorporates suggestions and/or hypnotic anchors that were imparted to the subject during the hypnosis session. A posthypnotic process is usually triggered by some event or circumstance as defined during a hypnotic session with a therapist. As described here, there are at least two exemplary categories of posthypnotic process that generally involve triggers which occur after the onset of an episode of a psychogenic affliction. Other categories of post hypnosis processes are possible, some that involve an onset of an affliction and some that have other triggers. Of the two embodiments of posthypnotic process categories focused on herein, one relates to identifying the cause of a psychogenic affliction and one relates to actions that mitigate the affliction.

A trigger typically results directly from an onset of an episode of an affliction, and typically occurs when the subject first realizes the affliction has begun or where the intensity of the unpleasantness of the affliction has reached a point where it has become a focus of their attention. It is also possible that while the subject has not consciously noticed the onset of the affliction, their unconscious mind has. Therefore, the trigger event that results from noticing an onset of an affliction may be noticed by either of the subject's conscious or unconscious mind. Likewise, the painful thoughts that caused the affliction may actually be conscious or unconscious. If unconscious, only a method like hypnosis or a hypnosis-induced posthypnotic process can access those thoughts to surface the related issues and allow mitigation of the affliction.

According to the invention, a posthypnotic process triggered by an onset of a psychogenic affliction may additionally be focused on mitigation of an affliction and include specific actions performed by a subject in addition to the subject thinking back in time—either consciously or unconsciously—and locating painful thoughts. To effect mitigation, the painful thoughts are then processed by the subject's mind, either consciously or unconsciously, according to a posthypnotic process previously imparted to them during a hypnotic session. Subsequently, based on results of processing the painful thoughts, a posthypnotic process aimed at affliction mitigation then may include any of the following exemplary and non-limiting actions, either singly or in combination, that include the subject:
unconsciously remembering the painful thoughts;
consciously remembering the painful thoughts;
recording the painful thoughts;
recalling a comforting phrase (either consciously or unconsciously);
recalling a phrase of resolve (either consciously or unconsciously);
recalling a phrase of acceptance (either consciously or unconsciously);
instructing their body to terminate the affliction (either consciously or unconsciously);
instructing their body to increase circulation in an afflicted area of their body (either consciously or unconsciously);
reminding them self that the affliction is generated by their mind and is not caused by a physical anomaly or physical stimulus (either consciously or unconsciously);
reminding them self that they can face a cause of the painful thoughts without the affliction (either consciously or unconsciously);
reminding them self to push beyond a previously established limit of physical activity (either consciously or unconsciously);
visualizing (either consciously or unconsciously) a rush of blood flow to the area of the body where a physical affliction is manifested; and
recalling an anchor image and associated feelings previously established under hypnosis;

Anchors and Post-Hypnotic Suggestions

The concept of post-hypnotic suggestion is well known. Anyone who has seen a demonstration of “stage hypnosis” has been exposed to the basic concept, although in practice stage hypnosis does not accurately represent how professional hypnotherapists work with their patients. In a very general sense, a suggestion is made under hypnosis including a trigger event and a resultant action by the subject. When the trigger event occurs sometime after the hypnosis session, the subject carries out the action or the suggestion influences the behavior or emotional state of the subject in some way. As some experts describe it, a post-hypnotic suggestion operates at a psychological level, whereas a hypnotic anchor operates at more of a neurological level in the unconscious mind. Anchors have been likened to a Pavlovian-like response mechanism between a trigger and a result. A post-hypnotic suggestion is imparted to a subject as a literal instruction or suggestion and is understood literally by the subject. An anchor operates at a much lower level without a conscious understanding by the subject. Anchors are created using different techniques than post-hypnotic suggestions and much discussion on how to create anchors exists in available literature.

A hypnotic anchor is a base level mechanism representing a link between a trigger and a response or resultant state. Setting anchors can be done intentionally or covertly; and is a process where the practitioner observes emotions or states of minds, and then sets the trigger, a special action or series of actions, that can be used later to invoke the same “state” or response. The effect is to influence feelings, states of consciousness, or habits, to come and go.

Anchoring in hypnosis may not accomplish direct control over some subjects and may be frequently used for indirectly controlling in combination with post-hypnotic suggestion. A hypnotic anchor may be used to reinforce or facilitate the effect of a post-hypnotic suggestion, such as to establishing feelings of confidence, resolve, wellbeing, or acceptance as the subject carries out actions to mitigate their affliction. An anchor can help create a “safe place” for a subject. Since anchors operate mostly at the unconscious level, it is frequently useful to include imagery or icons in establishing an anchor. A particular image may assist a subject in feeling safe, feeling more confident, or feeling more determined to name a few possibilities. Upon a trigger event, a subject may visualize an image in parallel with other actions that comprise a posthypnotic process.

In creating a posthypnotic process according to the invention, a therapist will craft a process according to an individual subject in order to best accomplish mitigation of an affliction including uncovering the cause of the affliction. In crafting this process they will typically use post-hypnotic suggestions, hypnotic anchors, or a combination of the two.

### BRIEF DESCRIPTION OF THE DRAWINGS

FIG. 1 shows a timeline describing a typical psychogenic process that a subject may experience including the onset of an episode of a chronic affliction.

FIG. 2 shows a flow diagram including an exemplary hypnotherapy session according to the invention, plus a follow-up therapy session occurring after a posthypnotic process according to the invention has occurred.

FIG. 3 shows a timeline including an onset of an episode of an affliction and an exemplary posthypnotic process activated by a trigger event according to the invention, including a follow-up therapy session.

FIG. 4 shows a timeline describing the onset of an episode of an affliction including affliction mitigation resulting from an exemplary posthypnotic process according to the invention.

### DETAILED DESCRIPTION

It is generally understood that psychogenic afflictions exist, and their study forms the basis of psychosomatic medicine. Psychogenic afflictions serve the purpose of diverting a subject's thoughts away from thoughts they find painful. As such, a subject frequently is not aware of what they were thinking just prior to the onset of an affliction—so thoroughly does the body's psychosomatic mechanism work. Processes are disclosed herein whereby a psychotherapist uses hypnosis to uncover painful thoughts that trigger psychogenic afflictions. According to the invention, posthypnotic processes are imparted to a subject under hypnosis such that painful thoughts a subject subsequently has immediately preceding the onset of a psychogenic affliction are uncovered. The painful thoughts may be immediately written down by the subject or remembered, either consciously or unconsciously, to be revealed later to a therapist. The posthypnotic process may also include thoughts and actions that a subject is encouraged to use in mitigating the affliction.

When it is suggested that the source of a person's pain is mind-related, most people quickly withdraw or overtly state that they want no part of such discussions. Their mind is a very sensitive subject for most people. Most people would never consider seeing a psychiatrist lest someone judge them as mentally unstable or even crazy. Hypnosis, on the other hand, brings another dimension to the process. If a subject believes that as a result of hypnosis they will be cured, some people will find that more palatable than engaging a therapist specifically to venture directly into the problems that trouble their mind. If under hypnosis they can face and deal with the issues causing emotional pain, then the process may seem less threatening. Also, people seem more amenable to the idea of a mindbody connection when presented with the concept that “stress” has exacerbated their physical affliction. For instance, it is generally acknowledged and accepted that stress causes ulcers, and some people will even admit that stress may contribute to their back pain. Therefore, entering a program based on hypnosis where they are told the issues related to their “stress” will be dealt with will seem less threatening to some people. Additionally, to the extent that the painful thoughts that cause their affliction can be faced and/or resolved unconsciously, a subject may find the overall therapeutic process less disturbing to their conscious mind.

As stated earlier, no one should assume that his or her symptoms are psychologically caused until a physician has ruled out the possibility of serious or life-threatening disease. Therefore it advisable for a therapist to initially check a subject's medical records to validate that no physical condition exists whereby delaying treatment by starting a psychotherapeutic process might do harm to the subject. Also, by verifying that a chronic affliction is most probably not caused by a physical phenomenon, a therapist is able to reinforce that reality in the subject—a fact that greatly facilitates the subject's ability to accept that their affliction is psychogenic. As suggested by Dr. Sarno and validated in practice, physical therapy focuses attention on a supposed physical anomaly and when the affliction is psychogenic chronic pain, this focus only serves to perpetuate the affliction. As Dr. Sarno suggests, discontinuing a physical therapy program while working through the psychogenic issues can be critical to achieving a positive outcome.

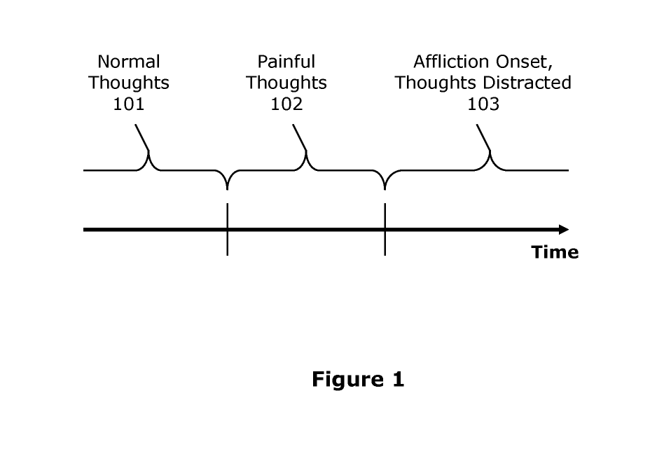

FIG. 1 shows a time line describing a typical psychogenic process that a subject may experience including the onset of an episode of a chronic affliction. The timeline begins with the subject going about their daily business and thinking normal thoughts 101. At some point in time, their thoughts stray to some form of painful thoughts 102, which can happen either consciously or unconsciously or some combination of the two. If the subject is prone to psychogenic afflictions, then it would not be atypical for an affliction onset 103 to occur shortly thereafter.

An onset of an episode of an affliction is when the subject first realizes the affliction has begun or where the intensity of the unpleasantness of the affliction has reached a point where it has become a focus of their attention. It is also possible that while the subject has not consciously noticed the onset of the affliction, their unconscious mind has. Therefore, a trigger event that results from noticing an onset of an affliction may be noticed by either of the subject's conscious or unconscious mind. Likewise, the painful thoughts may actually be conscious or unconscious. If unconscious, only a method like hypnosis or a hypnosis-induced posthypnotic process can access these thoughts to surface the related issues and mitigate the affliction.

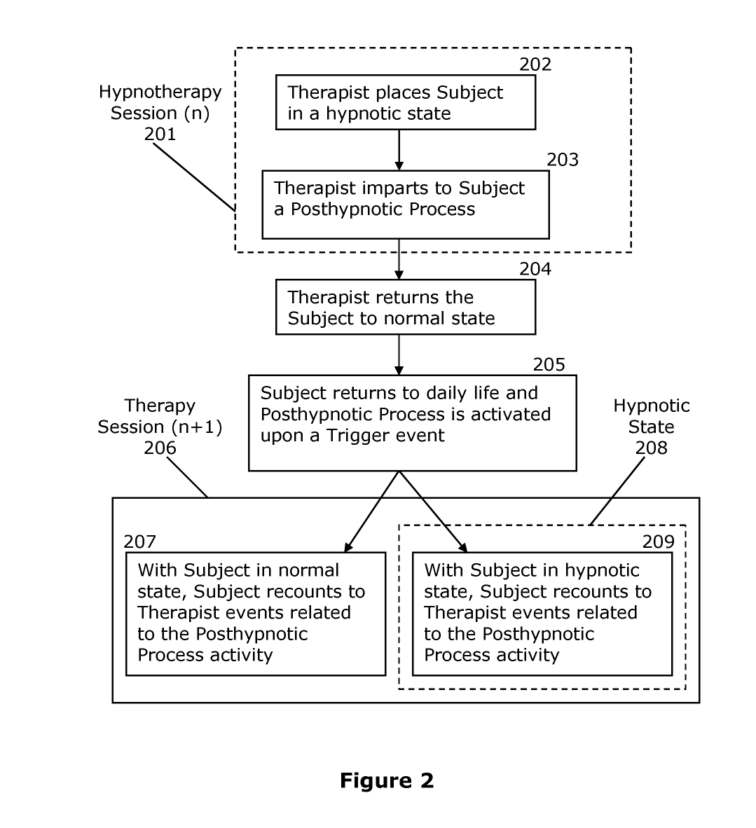

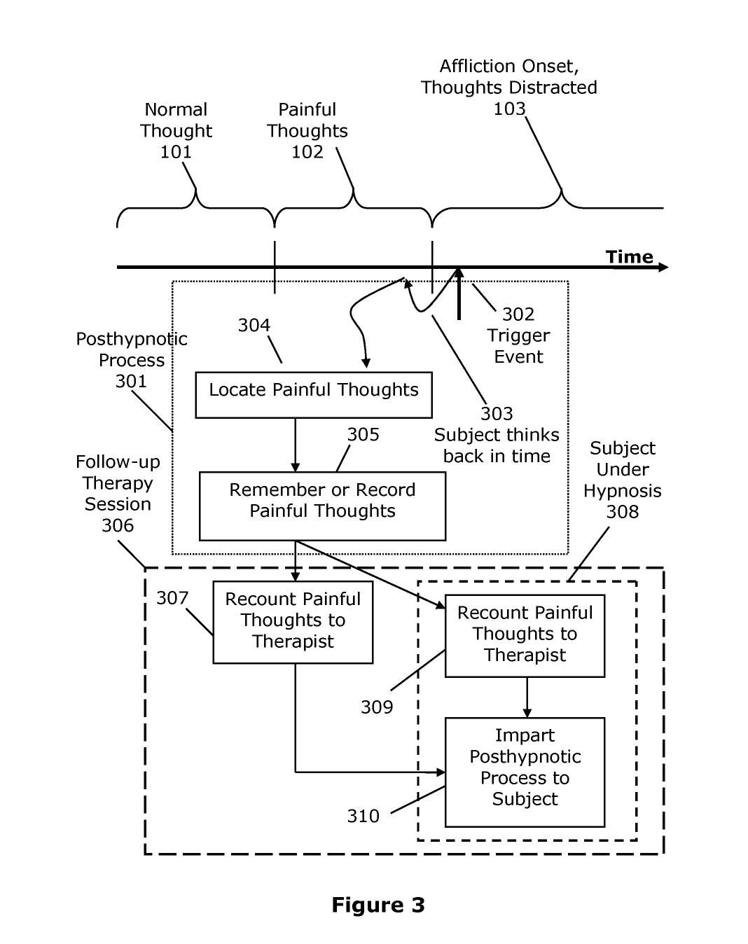

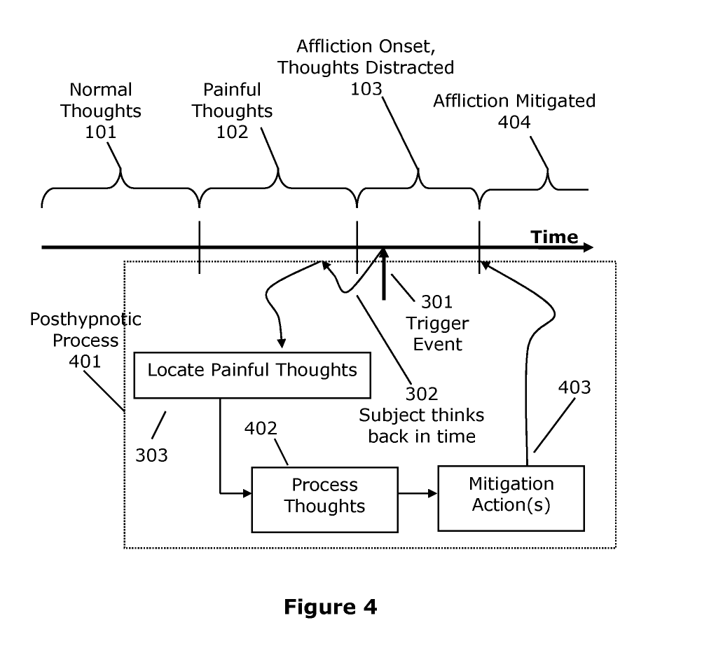

FIG. 2 shows a flow diagram including an exemplary hypnotherapy session according to the invention, plus a follow-up therapy session occurring after a posthypnotic process according to the invention has occurred. In a first hypnotherapy session, session (n) 201, a therapist initially places 202 a subject in a hypnotic state. Subsequently, the therapist imparts 203 a posthypnotic process to the subject. A posthypnotic process is any process involving a subject that occurs after a hypnosis session with the subject according to the invention, and relates to suggestions and/or hypnotic anchors imparted to the subject during the hypnosis session. A posthypnotic process is usually triggered by some event or circumstance as defined during a hypnotic session with a therapist. As described herein there are at least two exemplary categories of posthypnotic process that involve triggers that occur after the onset of an episode of an affliction. Other post hypnosis processes are possible, some that involve an onset of an affliction and some that have other triggers. Of the two embodiments of posthypnotic processes focused on herein, and as described with respect to FIGS. 3 and 4, one process relates to identifying the cause of a psychogenic affliction and one relates to actions that mitigate an affliction.

As shown in FIG. 2, at the completion of a hypnotherapy portion of a therapy session, the therapist returns 204 the subject to a normal state, and subsequently the subject returns 205 to daily life where at some later point in time the posthypnotic process is activated upon the occurrence of a trigger event. At yet a later point in time, a follow-up therapy session, session (n+1) 206 occurs. The follow-up therapy session may take place with the subject either in a hypnotic state 208 or in a normal state. When the follow-up therapy session is conducted with the subject in a normal state, the subject recounts 207 to the therapist events that transpired during the posthypnotic process. Alternately, if the follow-up therapy session is conducted with the subject in a hypnotic state 208, the subject will recount 209 under hypnosis events that transpired during the posthypnotic process. As with both conventional psychotherapy sessions and many hypnotherapy sessions, the follow-up session may be recorded and replayed for the subject after the subject is no longer in a hypnotic state.

FIG. 3 shows a time line including an onset of an episode of an affliction and an exemplary posthypnotic process activated by a trigger event according to the invention, including a follow-up therapy session. After a subject experiences the onset 103 of an affliction resulting from painful thoughts 102, posthypnotic process 301 begins with a trigger event 302. According to an exemplary posthypnotic process a subject then thinks back in time 303 to locate 304 the painful thoughts that resulted in the affliction. The act of “thinking back” may occur consciously or unconsciously, or through a combination of both. Once the painful thoughts have been located, the subject remembers or records 305 the painful thoughts such that they may later be made available in follow-up session 306 with a therapist. If the painful thoughts were located unconsciously, the act of remembering also occurs unconsciously. In reality, although a subject is most often distracted from their painful thoughts by the onset of an affliction, their unconscious mind maintains a record of their thoughts prior to the trigger event and subsequent distraction due to the onset of the affliction.

In follow-up therapy session 306, the subject may be in a normal state when they recount 307 their experiences of the posthypnotic process and any painful thoughts that were located. If they recorded the painful thoughts through any mechanism such as writing down the thoughts or verbally recording the thoughts, the recorded thoughts are supplied to a therapist during follow-up session 306. If during follow-up session 306 the subject is placed under hypnosis 308 in order to access the results of posthypnotic process 301, then they will under hypnosis recount 309 the painful thoughts to the therapist. Regardless of the state of the subject as they recount their experience during the posthypnotic process, the therapist may wish to impart a modified posthypnotic process to the subject and/or a new posthypnotic process. To do so, the subject is placed under hypnosis 308 and the new or modified posthypnotic process is imparted 310 to the subject.

Accessing and mitigating the causes of chronic afflictions per the methods described herein may frequently be an iterative process requiring modification to posthypnotic processes or the imparting of new and different posthypnotic processes to a subject. For instance, if the methods utilized in an initial posthypnotic process have not provided for accurate locating of the painful thoughts producing an affliction, a revised posthypnotic process may be utilized in which case a posthypnotic suggestion and/or a hypnotic anchor may be altered or replaced. Another purpose to impart a different posthypnotic process to the subject involves the circumstance where the different posthypnotic process has the purpose of mitigating the affliction rather than simply locating the cause of the affliction. Such a posthypnotic process aimed at affliction mitigation is described with respect to FIG. 4.

FIG. 4 describes a posthypnotic process including actions that are imparted to a subject as part of the posthypnotic process such that these actions have the effect of mitigating the subject's chronic affliction. Here, as part of posthypnotic process 401, trigger event 301 again causes a subject to search back in time 302 and locate painful thoughts 303. Subsequently the subject processes 402 those thoughts according to the process previously imparted to them under hypnosis by a therapist, and depending on the results of processing 402 the painful thoughts, takes certain actions 403 to mitigate the affliction. As a result, sometime after trigger event 301, these mitigation actions 403 will have taken effect and to some extent, the affliction suffered by the subject will have been mitigated 404.

The actions which a user may be encouraged to take according to the posthypnotic process will depend upon the nature of the painful thoughts and the subject's life situation with respect to elements of the circumstances causing the painful thoughts. The actions may take the form of a mental process, conscious or unconscious, to address the cause of the painful thoughts, and/or may take the form of actions that attempt to directly mitigate the physical nature of the affliction where the affliction has a physical component. For instance, it has been identified by Dr. Sarno and other researchers that for many cases of the mindbody affliction he has generically termed TMS, the physical mechanism creating psychogenic pain and many other psychogenic afflictions is frequently a reduction in circulation in the affected area of the body where the affliction is exhibited. As such, one exemplary posthypnotic suggestion directed to mitigate a psychogenic affliction may include a subject visualizing a rush of additional blood circulation flow in the affected localized area. This has proven to be especially useful in cases of psychogenic joint pain, dermatitis conditions like rosacea, and for treating a sudden onset of a migraine when applied during the very early stages where a subject is just beginning to see the “lights”.

The causes of painful thoughts which are the source of a psychogenic affliction are many and varied. For example these may include a painful family related situation, a painful work related situation, or a painful health related situation, to name a few. With regard to resolution, the causes of painful thoughts generally fall into three categories:
1) something the subject absolutely cannot change:
2) something the subject can change if they resolve to change it; and
3) something the subject may be able to change, however they cannot yet determine the probability of changing

The third of these causes is of course the most difficult to deal with.

Where the circumstance causing painful thoughts can clearly be either changed or not changed, a posthypnotic process can include the subject reminding them self that they can either change the situation causing the painful thoughts or alternately accept the situation that is the cause. This is similar to the message contained in the “Serenity Prayer”. The original Serenity Prayer is attributed to Niebuhr and translated into English reads as follows, with emphasis added regarding key elements that a posthypnotic process would typically focus on:
God, give us grace to accept with serenity the things that cannot be changed, Courage to change the things which should be changed, and the Wisdom to distinguish the one from the other. Living one day at a time, Enjoying one moment at a time, Accepting hardship as a pathway to peace, Taking, as Jesus did, This sinful world as it is, Not as I would have it, Trusting that You will make all things right, If I surrender to Your will, So that I may be reasonably happy in this life, And supremely happy with You forever in the next. Amen.

A non-religious and pro-active version of this statement that a subject could consciously or unconsciously repeat to them self, in the form of a re-affirming pledge, would be:
I will Accept with serenity the things that cannot be changed, I will have the Courage to change the things which should be changed, and I will have the Wisdom to distinguish one from the other.

How a phrase of resolve or a phrase of acceptance based on this passage is imparted within a posthypnotic process would be applied or personalized to each subject while taking their situation into account—including whether an affirmation should or should not have religious overtones. If it is discovered by a therapist in the process of FIG. 3 that a subject who has late stage terminal cancer is generating pain to distract their thoughts from dwelling on the inevitable outcome, then a posthypnotic process including repeating a phrase of acceptance may be the most appropriate course.

If however a subject has early stage cancer with psychogenic pain caused by similar painful thoughts, but is in a cancer treatment program where mindbody techniques are used to fight the cancer, then a phrase of resolve may be a better course and may also assist the program focused on fighting the cancer. In this case, a posthypnotic process may also include suggestions that invoke the mind and/or visualization techniques to attack the cancer cells.

Many adaptations or elaborations are possible on general phrases of resolve or acceptance. For instance a phrase of resolve might also include that a subject:

i) is currently confronting the problem

ii) has a plan to confront the problem

iii) will make a plan to confront the problem

And, for instance a phrase of acceptance might also include that a subject:

iv) cannot possibly alter or eliminate the problem and therefore must reach contentment with that reality

Cancer Patients

It is common for terminal cancer patients to have great physical pain. Most physicians attribute this pain to the physical deterioration of the body as caused by the cancer, whereas in many cases the pain is in fact psychogenic. For instance one exemplary late stage cancer patient had severe shoulder pain in one shoulder but months later the pain had moved to the other shoulder. This is very consistent with how the mind creates psychogenic affliction, sometimes hunting around the body for different places to effect a new mind-distracting affliction.

Variations on phrases of acceptance for a subject dying of late-stage, non-reversible cancer might for example include:
What I am moving toward is an eventuality for everyone—I just know the timing whereas some folks do not.
I can't change what is happening—so I will accept it and be at peace with it.
I'm tired, and dying will bring an eternal peace and rest.
(religious) Dying will bring an eternal life in a better place.
My family and friends are suffering as they watch me decline. They will be in less pain if I am in less pain. To that end, I resolve to be at peace with dying so my pain will end.

Amputees and Paraplegics

Recent wars have produced a disproportionate number of amputees due to IEDs combined with advances in field and trauma medicine. An amputee may experience psychogenic afflictions as their mind copes to deal with the painful thoughts related to their loss. A combination of phrases of acceptance and resolve may be appropriate—acceptance of the loss of a limb or limbs, and resolve to move ahead with solutions that move them closer to a normal life. Likewise, a paraplegic may be benefited by similar phrases of acceptance and resolve as part of a posthypnotic process.

Depression

To a great extent, chronic depression is commonly thought of and treated as a chemical imbalance in the brain. Certain drugs may alleviate the symptoms for many subjects, however some drugs have significant side affects. Many drugs for treating depression even caution against a result including worsened depression leading to suicidal thoughts. While there may be physical causes of depression, psychogenic causes may also be at work based on painful thoughts that occur either consciously or unconsciously. A posthypnotic process can help to reveal psychological causes of depression and subsequently a posthypnotic process including mitigation actions may assist a therapist in treating a subject's depression.

PTSD

As in most wars over the centuries, the recent wars have produced many cases of PTSD (Post Traumatic Stress Disorder). The difference is that in current thinking, this condition is accepted and treated. When a subject with PTSD experiences a chronic mental or physical affliction, that affliction may at least in part be psychogenic due to painful thoughts with respect to memories of their war experiences. The methods described herein may be used to treat and mitigate a psychogenic affliction resulting from PTSD, based on discovering the specific nature of the painful thoughts.

Adaptation of the Posthypnotic Process to Individual Subjects

Psychotherapy by its nature is iterative and typically involves a series of sessions with a subject. In many cases the sessions are recorded as a record for the therapist and/or to allow the subject to play back the session at a later time. By reviewing the session, a subject can better understand the process and may be more likely to accept certain aspects of their therapy and the realities of how their mind and body interact.

According to the psychotherapy process described herein, a therapist may optionally choose to monitor a subject during their therapy sessions using electronic monitoring devices such as for instance electroencephalographs and blood pressure monitors to document the degree of a subject's hypnotic state and the subject's physical and mental condition and reactions while under hypnosis as well as when in a normal state. Readings taken may be recorded and compared with readings taken during other sessions of the subject. Readings may also be compared with those of other subjects undergoing similar therapy strategies. Electronic monitoring may be used to gauge a subject's body response including the depth of a hypnotic trance and their response to receiving a posthypnotic process including their response to imparted suggestions and imparted anchors. In follow-up sessions, electronic monitoring can gauge a subject's response to recounting painful thoughts as well as their response to recounting mitigation actions. It is also useful to gauge a subject's response to questions regarding their belief and acceptance that their affliction is psychogenic and not physically caused.

Pushing Past Artificial Boundaries

A subject who has lived with chronic pain for a considerable time is likely to have established artificial barriers in their life in their attempt to avoid initiating an onset of their affliction, or out of fear of causing further injury to them self and thereby prolonging or intensifying their pain. For instance, many sufferers of chronic back pain for the reasons just enumerated develop a fear of performing certain actions, including for example: sitting in certain styles of chairs; standing in line; bending over; tying their shoes; and lifting objects. Over time, a subject who behaves in this manner creates mechanisms that essentially “box them in” to an artificial world of their own creation and one that is essentially abnormal, and also that help to perpetuate a chronic affliction. Part of a treatment for mitigating chronic afflictions of such subjects is to encourage the subject as part of a posthypnotic process to push through artificial boundaries when confronted with situations that they feel may trigger acute outbreaks of an affliction. A posthypnotic process that includes pushing past artificial boundaries may or may not include a location of painful thoughts 102 as described per FIG. 4. However when used in conjunction with a process such as that of FIG. 4, removing artificial boundaries can help facilitate mitigation of the psychogenic mechanism producing the affliction.

Reinforcing the Nonphysical Cause

As mentioned earlier, it is useful in the early stage of a treatment plan according to the processes described herein to enumerate with the subject their history of mindbody afflictions throughout their life. Many of these will be afflictions that the subject can easily agree with and accept as caused by their mind such as: indigestion and ulcers; a “nervous” stomach, high blood pressure upon being upset or angered, and certain skin conditions such as hives. In the case of chronic joint or back pain, in current times there are often MRI images available to a subject that show some physical abnormality. In most cases the subject is told that according to conventional medicine their pain is due to the abnormality. As part of a diagnosis for back pain, the subject is frequently told that they have “degenerative disc disease”, which in addition to causing great distress certainly helps to reinforce that the cause of their pain is physical. In fact, everyone's disks degrade over their life. Changes that occur with age include loss of fluid in the spinal discs, resulting in thinner discs which narrows the gap between the vertebrae. The loss in fluid also makes the discs less flexible. The overall effect is a loss in the shock absorbing properties of the discs. Aging can also result in cracks or tears occurring in the outer layer of the spinal discs that result in leaking of the viscous liquid from inside the spinal discs. To varying extents this process happens to everyone, however most people do not have great pain as a result of this natural process.

Even if a subject is inclined to accept that they are prone to mindbody afflictions by examining their medical history over their life, it may still be hard for them to accept that the cause of their back or joint pain is psychogenic after being shown MRI images of physical anomalies supposedly causing their pain. They may at times during their treatment be haunted by the physical anomaly they were previously told was the cause. It may therefore be useful during a hypnotic session to provide background information to a subject that reinforces the psychogenic nature of their affliction based on research in the field and the experiences of others.

Numerous studies exist that prove the reality of mindbody phenomenon with respect to afflictions such as joint and back pain. For instance in one study MRIs were performed for purposes other than examining the vertebrae of the back, however at the same time coincidentally scanned the area of the lower back where spinal deterioration is typically observed. These subjects had no back pain at the time the MRIs were taken. However, it was found that consistent with the aging process, the older patients had progressively more disk degeneration, and in many cases had visible disk degeneration at a similar level to other patients not in the study who experienced extreme back pain. Other studies also show that the peak age groups for chronic back pain are between the mid-30s and mid-50s, essentially the peak years of career activity and family rearing for most people, and therefore the time frames during which the greatest amount of stress occurs. To reinforce for a subject with psychogenic pain that the cause of their pain is not physical, it may be useful during a therapy session and with the subject in a hypnotic state, to recount to the subject various studies such as these that help reinforce the nonphysical nature of the cause of the affliction. It may also be useful to recount for the subject specific case studies of individual subjects where an acceptance and understanding of the psychogenic nature of an affliction has helped to mitigate the affliction. Group therapy has been shown to be useful for treating mindbody disorders as different sufferers recount to other sufferers how they dealt with their afflictions. While group therapy may not be practical with respect to a hypnotherapy process, providing a subject access to evidence of how the processes described herein have helped others may be particularly useful.

The foregoing description of preferred embodiments of the present invention has been provided for the purposes of illustration and description. It is not intended to be exhaustive or to limit the invention to the precise forms disclosed. Many modifications and variations will be apparent to one of ordinary skill in the relevant arts, while remaining within the scope of the appended claims. For example, steps preformed in the embodiments of the invention disclosed can be performed in alternate orders, certain steps can be omitted, and additional steps can be added. The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalents.

What is claimed is:

1. A psychotherapeutic process for mitigating a chronic affliction in a subject, comprising:
placing the subject in a hypnotic state; and
while the subject is in the hypnotic state, imparting to the subject a posthypnotic process responsive to a trigger event, whereby the subject's actions according to the posthypnotic process comprise:
upon the onset of the trigger event, locating painful thoughts preceding the trigger event;
remembering or recording the painful thoughts preceding the trigger event; and
wherein the trigger event is associated with an onset of an episode of the affliction.

2. The psychotherapeutic process of claim 1 wherein imparting to the subject a posthypnotic process is implemented by way of one or both of a post-hypnotic suggestion and establishing a hypnotic anchor.

3. The psychotherapeutic process of claim 1 wherein locating the painful thoughts preceding the trigger event is performed consciously by the subject.

4. The psychotherapeutic process of claim 1 wherein locating the painful thoughts preceding the trigger event is performed unconsciously by the subject.

5. The psychotherapeutic process of claim 3, further comprising:
holding a follow-up session including a therapist and the subject, wherein the subject is fully conscious;
during the follow-up session, requesting that the subject recount the painful thoughts that preceded the trigger event and that the subject consciously located; and
discussing with the subject, the significance of the painful thoughts with respect to the onset of the affliction.

6. The psychotherapeutic process of claim 4, further comprising:
holding a follow-up session including a therapist and the subject;
during the follow-up session, placing the subject in a hypnotic state;
with the subject in the hypnotic state, requesting that the subject recount the painful thoughts that preceded the trigger event and that the subject unconsciously located; and
placing the subject in a fully conscious state, and discussing with the subject the significance of the painful thoughts with respect to the onset of the affliction.

7. The psychotherapeutic process of claim 1, further comprising:
having a follow-up session including a therapist and the subject, wherein the subject is fully conscious;
reviewing with the subject, the painful thoughts that preceded the trigger event and that the subject had recorded; and
discussing with the subject, the significance of the painful thoughts preceding the trigger event with respect to the onset of the affliction.

8. A psychotherapeutic process for mitigating a chronic affliction in a subject, comprising:
placing the subject in a hypnotic state; and
while the subject is in the hypnotic state, imparting to the subject a posthypnotic process responsive to a trigger event, whereby the subject's actions according to the posthypnotic process comprise:
upon the onset of the trigger event, locating painful thoughts preceding the trigger event;
processing the painful thoughts;
acting based on a result of the processing of the painful thoughts; and
wherein the trigger event is associated with an onset of an episode of the affliction.

9. The psychotherapeutic process of claim 8 wherein the subject's actions according to the posthypnotic process include the subject recalling a comforting phrase; and
wherein the subject recalling a comforting phrase is performed either consciously or unconsciously by the subject.

10. The psychotherapeutic process of claim 8 wherein the subject's actions according to the posthypnotic process include the subject recalling a phrase of acceptance; and
wherein the subject recalling a phrase of acceptance is performed either consciously or unconsciously by the subject.

11. The psychotherapeutic process of claim 8 wherein the subject's actions according to the posthypnotic process include the subject recalling a phrase of resolve; and
wherein the subject recalling a phrase of resolve is performed either consciously or unconsciously by the subject.

12. The psychotherapeutic process of claim 8 wherein the subject's actions according to the posthypnotic process include the subject instructing their body to terminate the affliction; and
wherein the subject instructing their body to terminate the affliction is performed either consciously or unconsciously by the subject.

13. The psychotherapeutic process of claim 8 wherein the subject's actions according to the posthypnotic process include the subject instructing their body to increase circulation in an afflicted area of their body; and
wherein the subject instructing their body to increase circulation in an afflicted area of their body is performed either consciously or unconsciously by the subject.

14. The psychotherapeutic process of claim 8 wherein the subject's actions according to the posthypnotic process include the subject reminding them self that the affliction is generated by their mind and is not caused by a physical anomaly or physical stimulus; and
wherein the subject reminding them self that the affliction is generated by their mind and is not caused by a physical anomaly or physical stimulus is performed either consciously or unconsciously by the subject.

15. The psychotherapeutic process of claim 8 wherein the subject's actions according to the posthypnotic process include the subject reminding them self that they can face a cause of the painful thoughts without the affliction; and
wherein the subject reminding them self that they can face a cause of the painful thoughts without the affliction is performed either consciously or unconsciously by the subject.

16. The psychotherapeutic process of claim 8 wherein the subject's actions according to the posthypnotic process include the subject reminding them self to push beyond a previously established limit of physical activity; and
wherein the subject reminding them self to push beyond a previously established limit of physical activity is performed either consciously or unconsciously by the subject.

17. The psychotherapeutic process of claim 8 wherein the subject's actions according to the posthypnotic process include the subject visualizing a rush of blood flow to the area of the body where a physical affliction is manifested; and
wherein the subject visualizing a rush of blood flow to the area of the body where a physical affliction is manifested is performed either consciously or unconsciously by the subject.

18. The psychotherapeutic process of claim 8 wherein the subject's actions according to the posthypnotic process include the subject recalling an anchor image and associated feelings previously established under hypnosis.
